(19) United States
(12) Patent Application Publication
(10) Pub. No.: US 2015/0018817 A1
(43) Pub. Date: Jan. 15, 2015

(54) MULTIPLE ELECTRODE CONDUCTIVE BALLOON

(71) Applicant: BOSTON SCIENTIFIC SCIMED, INC., MAPLE GROVE, MN (US)

(72) Inventor: MARTIN R. WILLARD, BURNSVILLE, MN (US)

(21) Appl. No.: 14/316,432

(22) Filed: Jun. 26, 2014

Related U.S. Application Data
(60) Provisional application No. 61/845,281, filed on Jul. 11, 2013.

(51) Int. Cl. A61B 18/14; B29C 47/00

(52) U.S. Cl. CPC A61B 18/14; B29C 47/0026; A61B 2018/1472; A61B 2018/0022
USPC 606/41; 427/2.12; 264/129

(57) ABSTRACT

Medical devices and methods for making and using medical devices are disclosed. An example medical device may include a medical device for modulating nerves. The medical device may include an elongate shaft having a distal region. A balloon may be coupled to the distal region. An electrode may be disposed within the balloon. A virtual electrode may be defined along the balloon. The virtual electrode may include a region having a first non-conductive layer and a second conductive layer.

### CROSS-REFERENCE TO RELATED APPLICATIONS

This application claims priority under 35 U.S.C. §119 to U.S. Provisional Application Ser. No. 61/845,281, filed Jul. 11, 2013, the entirety of which is incorporated herein by reference.

### TECHNICAL FIELD

The present disclosure pertains to medical devices, and methods for manufacturing medical devices. More particularly, the present disclosure pertains to elongated medical devices for modulating nervous system activity.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a medical device for modulating nervous system activity. The medical device may include an elongated shaft having a distal region. A balloon may be coupled to the distal region. The balloon may have an inner non-conductive layer and an outer conductive layer. An electrode may be disposed within the balloon. A virtual electrode may be defined on the balloon. The virtual electrode may include a conductive region defined along a first portion of the balloon that is free of the inner non-conductive layer.

An example method for manufacturing a medical device may include providing an expandable balloon formed from a non-conductive material, forming one or more through holes in a portion of the expandable balloon, and applying a conductive material over the portion of the expandable balloon including the one or more through holes. A virtual electrode may be defined at a region adjacent to the one or more through holes.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments in connection with the accompanying drawings, in which:

FIG. 1 is a schematic view illustrating a renal nerve modulation system in situ;

FIG. 2 is a side view of a portion of an example medical device;

FIG. 3 is a cross-sectional view taken through line 3-3 in FIG. 2; and

FIGS. 4-6 illustrate some portions of an example method for manufacturing a medical device.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the invention. The illustrative embodiments depicted are intended only as exemplary. Selected features of any illustrative embodiment may be incorporated into an additional embodiment unless clearly stated to the contrary.

Certain treatments require the temporary or permanent interruption or modification of select nerve function. One example treatment is renal nerve ablation, which is sometimes used to treat conditions related to hypertension, congestive heart failure, diabetes, or other conditions impacted by high blood pressure or salt retention. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

While the devices and methods described herein are discussed relative to renal nerve modulation, it is contemplated that the devices and methods may be used in other treatment locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pulmonary vein isolation, tumor ablation, benign prostatic hyperplasia therapy, nerve excitation or blocking or ablation, modulation of muscle activity, hyperthermia or other warming of tissues, etc. In some instances, it may be desirable to ablate perivascular renal nerves with ultrasound ablation.

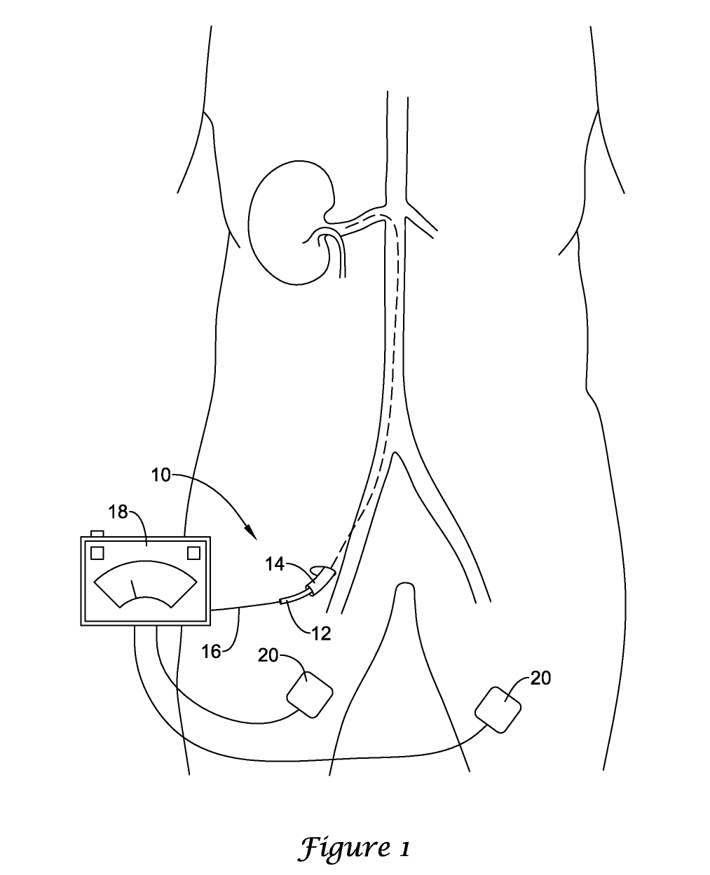

FIG. 1 is a schematic view of an illustrative renal nerve modulation system in situ. System 10 may include one or more conductive element(s) 16 for providing power to a renal ablation system including a renal nerve modulation device 12 and, optionally, within a delivery sheath or guide catheter 14. A proximal end of conductive element(s) 16 may be connected to a control and power unit 18, which may supply the appropriate electrical energy to activate one or more electrodes disposed at or near a distal end of the renal nerve modulation device 12. In addition, control and power unit 18 may also be utilized to supply/receive the appropriate electrical energy and/or signal to activate one or more sensors disposed at or near a distal end of the renal nerve modulation device 12. When suitably activated, the electrodes are capable of ablating tissue as described below and the sensors may be used to sense desired physical and/or biological parameters. The terms electrode and electrodes may be considered to be equivalent to elements capable of ablating adjacent tissue in the disclosure which follows. In some instances, return electrode patches 20 may be supplied on the legs or at another conventional location on the patient's body to complete the electrical circuit. A proximal hub (not illustrated) having ports for a guidewire, an inflation lumen and a return lumen may also be included.

The control and power unit 18 may include monitoring elements to monitor parameters such as power, voltage, pulse size, temperature, force, contact, pressure, impedance and/or shape and other suitable parameters, with sensors mounted along renal nerve modulation device 12, as well as suitable controls for performing the desired procedure. In some embodiments, the power unit 18 may control a radiofrequency (RF) electrode and, in turn, may “power” other electrodes including so-called “virtual electrodes” described herein. The electrode may be configured to operate at a suitable frequency and generate a suitable signal. It is further contemplated that other ablation devices may be used as desired, for example, but not limited to resistance heating, ultrasound, microwave, and laser devices and these devices may require that power be supplied by the power unit 18 in a different form.

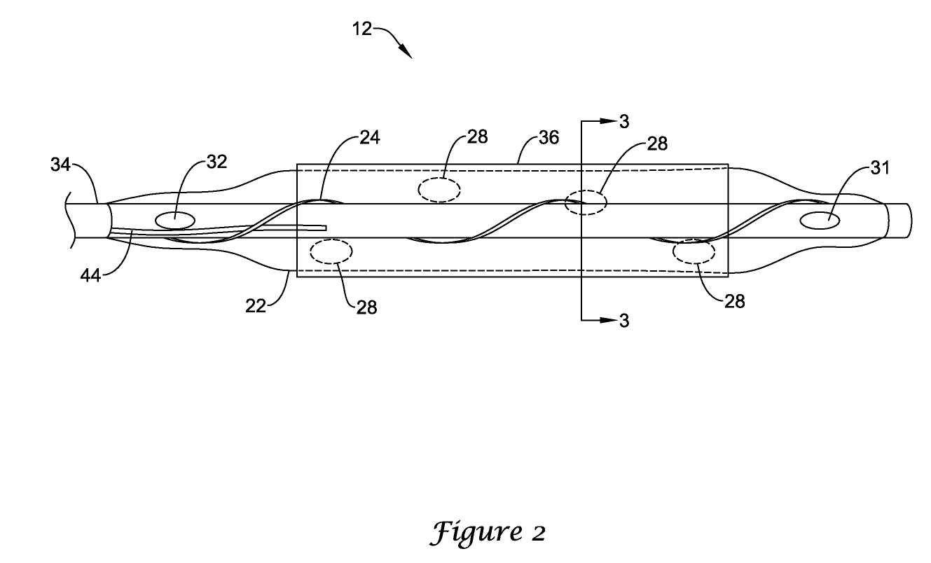

FIG. 2 illustrates a distal portion of a renal nerve modulation device 12. Here it can be seen that renal nerve modulation device 12 may include an elongate member or catheter shaft 34, an expandable member or balloon 22 coupled to shaft 34, and an electrode 24 disposed within balloon 22. Additional electrodes 24 may also be utilized. Balloon 22 may include an outer layer or sheath 36 positioned over a portion of the balloon 22. One or more sensors 44 (e.g., a thermistor, a thermocouple, or the like) may be included and may be disposed on the shaft 34, on the balloon 22 or at another suitable location.

When in use, balloon 22 may be filled with a conductive fluid such as saline to allow the ablation energy (e.g., radiofrequency energy) to be transmitted from electrode 24, through the conductive fluid, to one or more virtual electrodes 28 disposed along balloon 22. While saline is one example conductive fluid, other conductive fluids may also be utilized including hypertonic solutions, contrast solution, mixtures of saline or hypertonic saline solutions with contrast solutions, and the like. The conductive fluid may be introduced through a fluid inlet 31 and evacuated through a fluid outlet 32. This may allow the fluid to be circulated within balloon 22. As described in more detail herein, virtual electrodes 28 may be generally hydrophilic portions of balloon 22. Accordingly, virtual electrodes 28 may absorb fluid (e.g., the conductive fluid) so that energy exposed to the conductive fluid can be conducted to virtual electrodes 28 such that virtual electrodes 28 are capable of ablating tissue.

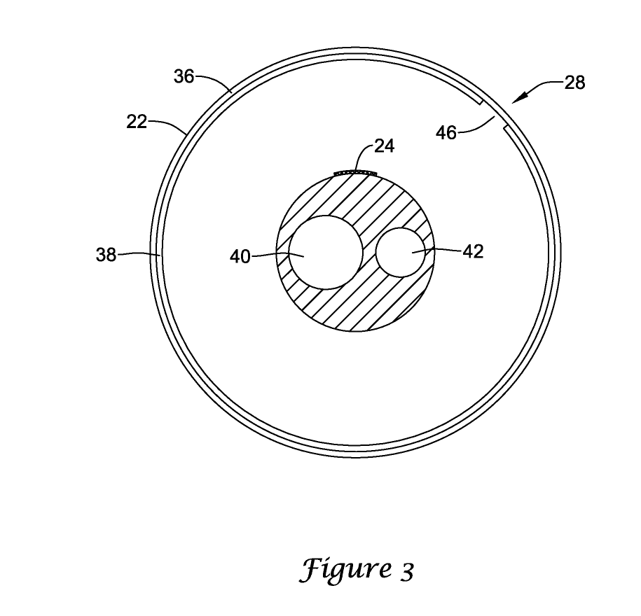

Referring briefly to FIG. 3, shaft 34 may include a guidewire lumen 40, a lumen 42 connected to the fluid inlet 31, and a lumen (not shown) connected to the fluid outlet 32. Other configurations are contemplated. In some embodiments, guidewire lumen 40 and/or one of the fluid lumens may be omitted. In some embodiments, guidewire lumen 40 may extend from the distal end of device 12 to a proximal hub. In other embodiments, the guidewire lumen can have a proximal opening that is distal the proximal portion of the system. In some embodiments, the fluid lumens can be connected to a system to circulate the fluid through the balloon 22 or to a system that supplies new fluid and collects the evacuated fluid. It can be appreciated that embodiments may function with merely a single fluid lumen and a single fluid outlet into the balloon. For example, in some instances, active cooling, or recirculation of the fluid, may not be necessary and a single opening may be used as both a fluid inlet and a fluid outlet

Electrode 24 (or a conductive element to supply power to electrode 24) may extend along the outer surface of shaft 34 or may be embedded within the shaft. Electrode 24 proximal to the balloon may be electrically insulated and may be used to transmit power to the portion of the electrode 24 disposed within balloon 22. Electrode 24 may be a wire filament electrode made from platinum, gold, stainless steel, cobalt alloys, or other non-oxidizing materials. These elements may also be clad with copper in another embodiment. In some instances, titanium, tantalum, or tungsten may be used. Electrode 24 may extend along substantially the whole length of the balloon 22 or may extend only as far as the distal edge of the most distal virtual electrode 28. The electrode 24 may have a generally helical shape and may be wrapped around shaft 34. While the electrode 24 is illustrated as having adjacent windings spaced a distance from one another, in some instances the windings may be positioned side by side. Alternatively, electrode 24 may have a linear or other suitable configuration. In some cases, electrode 24 may be bonded to shaft 34. The electrode 24 and virtual electrodes 28 may be arranged so that the electrode extends directly under the virtual electrodes 28. In some embodiments, electrode 24 may be a ribbon or may be a tubular member disposed around shaft 34. In some embodiments, a plurality of electrodes 24 may be used and each of the plurality may be fixed to the shaft 34 under virtual electrodes 28 and may share a common connection to conductive element 16. In other embodiments that include more than one electrode, each electrode may be separately controllable. In such embodiments, balloon 22 may be partitioned into more than one chamber and each chamber may include one or more electrodes. The electrode 24 may be selected to provide a particular level of flexibility to the balloon to enhance the maneuverability of the system. It can be appreciated that there are many variations contemplated for electrode 24.

A cross-sectional view of the shaft 34 distal to fluid outlet 32 is illustrated in FIG. 3. The guidewire lumen 40 and the fluid inlet lumen 42 are present, as well as electrode 24. In addition, balloon 22 is shown in cross-section as having an inner layer 38 and an outer layer 36. Virtual electrode 28 is formed in balloon 22 by the absence of inner layer 38. Inner layer 38 may extend the entire length of balloon 22 while outer layer 36 may extend along a portion of the length of balloon 22. Balloon 22 may be formed by extruding and molding a higher strength material, such as, but not limited to polyether block amide (e.g. PEBAX®, commercially available from Arkema headquartered in King of Prussia, Pa.). Other suitable materials include any of a range of electrically non-conductive polymers. These are just examples. The high strength material may form inner layer 38. Small holes 46 may be cut, or otherwise formed, through the inner layer 38 to form regions for virtual electrodes 28. A thin tube of a second material may be bonded to the outside of inner layer 38, covering holes 46, to form outer layer 36. It is contemplated that outer layer 38 may be formed in a number of different manners, such as, but not limited to extrusion, spraying, dipping, molding, etc. and may be attached to the inner layer 38 by thermal or adhesive methods. These are just examples. Outer layer 36 may include a hydrophilic, hydratable, RF permeable, and/or conductive material. One example material is hydrophilic polyurethane (e.g., TECOPHILIC® TPUs such as TECOPHILIC® HP-60D-60 and mixtures thereof, commercially available from the Lubrizol Corporation in Wickliffe, Ohio). Other suitable materials include other hydrophilic polymers such as hydrophilic polyether block amide (e.g., PEBAX® MV1074 and MH1657, commercially available from Arkema), hydrophilic nylons, hydrophilic polyesters, block co-polymers with built-in hydrophilic blocks, polymers including ionic conductors, polymers including electrical conductors, metallic or nanoparticle filled polymers, and the like. Suitable hydrophilic polymers may exhibit between 20% to 120% water uptake (or % water absorption) due to their hydrophilic nature or compounding. In at least some embodiments, outer layer 36 may include a hydratable polymer that is blended with a non-hydratable polymer such as a non-hydratable polyether block amide (e.g., PEBAX® 7033 and 7233, commercially available from Arkema) and/or styrenic block copolymers such as styrene-isoprene-styrene. These are just examples. Compounding a non-hydratable polymer with a hydratable polymer to form outer layer 36 may increase its strength. However, this is not required.

The materials of the inner layer 38 and the outer layer 36 may be selected to have good bonding characteristics between the two layers. It is further contemplated that the material of the inner layer 38 may be selected to provide a strong balloon 22. For example, a balloon 22 may be formed from an inner layer 38 made from a regular or non-hydrophilic polyether block amide and an outer layer 36 made from a hydrophilic polyether block amide. In other embodiments, a suitable tie layer (not illustrated) may be provided between adjacent layers. These are just examples. In some instances, the materials of the inner layer 38 and the outer layer 36 may be selected to have oriented material such that the inner and outer layers 38, 36 have similar stretch properties.

Prior to use, balloon 22 may be hydrated as part of the preparatory steps. Hydration may be effected by soaking the balloon in a saline solution. During ablation, a conductive fluid may be infused into balloon 22, for example via outlet 32. The conductive fluid may expand the balloon to the desired size. The balloon expansion may be monitored indirectly by monitoring the volume of conductive fluid introduced into the system or may be monitored through radiographic or other conventional means. Optionally, once the balloon is expanded to the desired size, fluid may be circulated within the balloon by continuing to introduced fluid through the fluid inlet 31 while withdrawing fluid from the balloon through the fluid outlet 32. The rate of circulation of the fluid may be between but not limited to 5 and 20 ml/min. This is just an example. The circulation of the conductive fluid may mitigate the temperature rise of the tissue of the blood vessel in contact with the non-virtual electrode areas. In some instances, it may not be necessary to circulate the conductive fluid.

Electrode 24 may be activated by supplying energy to electrode 24. The energy may be supplied at 400-500 KHz at about 5-30 watts of power. These are just examples, other energies are contemplated. The energy may be transmitted through the medium of the conductive fluid and through virtual electrodes 28 to the blood vessel wall to modulate or ablate the tissue. The inner layer 38 of the balloon prevents the energy transmission through the balloon wall except at virtual electrodes 28 (which lack inner layer 38).

Electrode 24 may be activated for an effective length of time, such as less than 1 minute, 1 minute, 2 minutes, or greater than 2 minutes. Once the procedure is finished at a particular location, balloon 22 may be partially or wholly deflated and moved to a different location such as the other renal artery, and the procedure may be repeated at another location as desired using conventional delivery and repositioning techniques. It is contemplated that virtual electrodes 28 may be formed at various locations along the length of the balloon 22 and various locations about the circumference of the balloon 22. This may allow for tissue modulation around an entire circumference of a vessel simultaneously. However, this is not required. The location(s) and number of virtual electrodes 28 may be varied as desired.

Disclosed herein are medical devices, balloons, and methods for making the same where one or more discrete balloon “virtual electrodes” are defined. The virtual electrodes are designed to reduce capacitive effects, thus reducing unwanted heating at non-electrode regions. In addition, the virtual electrodes are designed to include a strong balloon 22 having a reduced number of pin-hole problems due to its increased strength. Some of these and other features are described in more detail herein.

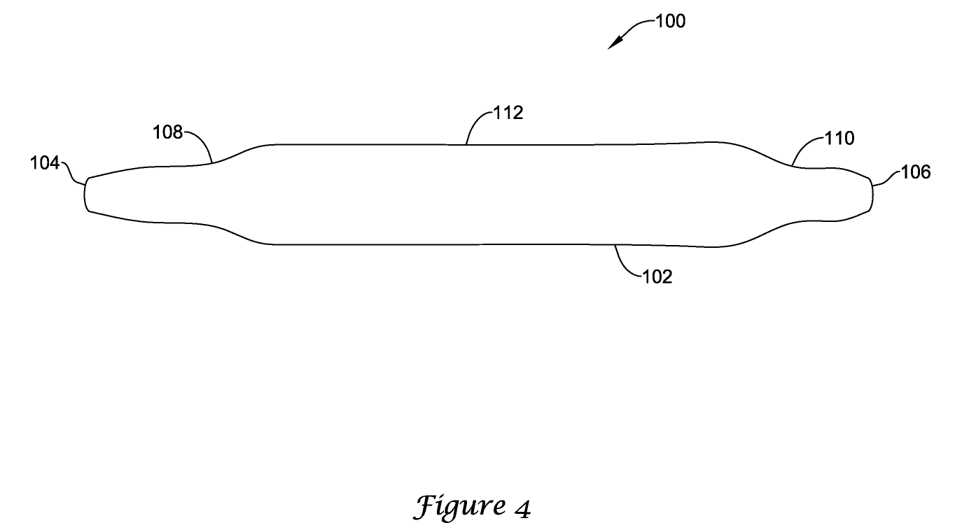

FIGS. 4-6 illustrate some portions of an example method for manufacturing an illustrative balloon 100. In general, the process may result in a balloon 100 having a region including by two layers and regions including by a single layer defining virtual electrodes. In the schematic drawings, other portions of the catheter or medical device that includes balloon 100 may not be seen. The other portions of the devices may or may not be present during the manufacturing process. The intent of showing these structures in the drawings is to demonstrate that balloon 100 may be used with medical devices such as those disclosed herein. In addition, balloon 100 may be utilized in medical devices such as device 12 (and/or other devices disclosed herein). Accordingly, the structural features of balloon 100 may be incorporated into device 12 (and/or other devices disclosed herein).

FIG. 4 is a side view of a portion of an example balloon 100. Balloon 100 may include a base or inner layer 102. Inner layer 102 may include a proximal end 104, a distal end 106, a proximal waist 108, a distal waist 110, and an intermediate region 112 disposed between the proximal and distal waists 108, 110. In at least some embodiments, inner layer 102 may include an electrically non-conductive high strength material such as those materials disclosed herein Inner layer 102 may be by casting, spraying, dipping, extrusion, molding, etc. In some embodiments, extruding a polymer tubing and then molding the polymer tubing into inner layer 102 may orient the material and provide increased strength over casting or spraying processes.

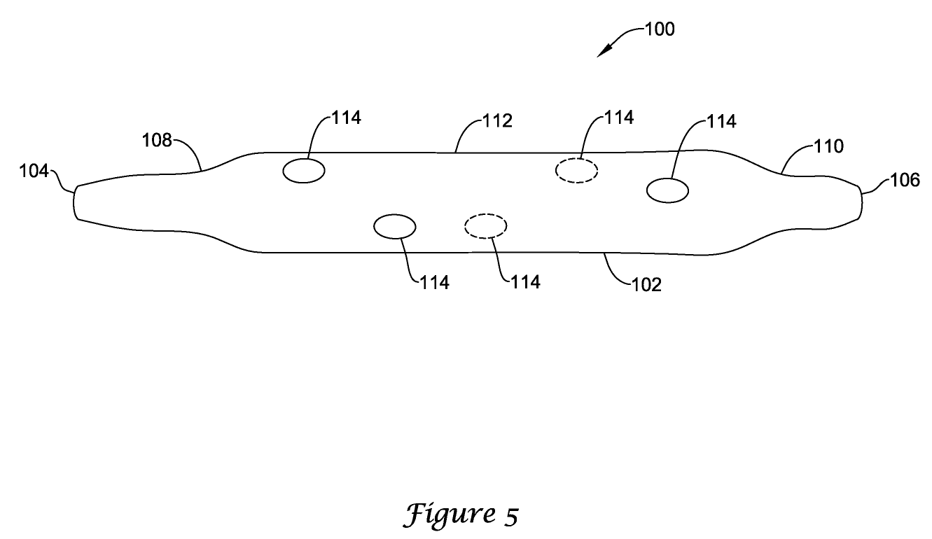

Once inner layer 102 has been formed, holes 114 may be formed through the inner layer 102, as illustrated in FIG. 5. Holes 114 may extend from an outer surface of inner layer 102 to an inner surface of inner layer 102 to define a through hole. While five holes 114 are illustrated, it is contemplated that the balloon 100 may include any number of holes desired, such as, but not limited to, one, two, three, four, or more. It is further contemplated that the holes 114 may take any shape desired, such as, but not limited to, circular, ovoid, square, rectangular, polygonal, etc. Holes 114 may be of any size desired to achieve the desired treatment. In some instances, holes 114 may be formed about the length and circumference of the intermediate region 112 in a helical pattern. However, this is not required. Holes 114 may be formed in any pattern or without a pattern, as desired. In some embodiments, holes 114 may extend proximally or distally of intermediate region 112. Holes 114 may be formed through any manner desired. For example, holes 114 may be drilled, punched, cut, laser formed, etched, etc. These are just examples. In some instances multiple small holes 114 (for example, but not limited to, in the range of 0.0005 inches to 0.010 inches in diameter) may be grouped together to form a virtual electrode. The holes 114 that make up the virtual electrode could be made in any desired pattern, e.g., a multiplicity of holes that make a spiral band around the balloon, or rings around the balloon 100. Small holes 114 could be made by, for example, by laser drilling.

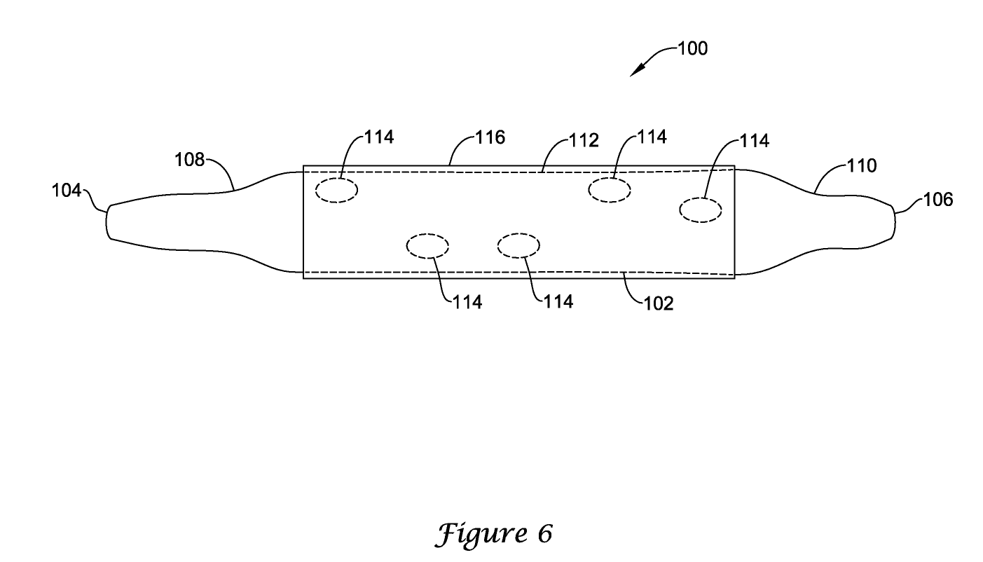

Once holes 114 have been formed, an outer layer 116 may be disposed over inner layer 102, as shown in FIG. 6. In at least some embodiments, outer layer 116 may include a hydrophilic and/or conductive material such as those materials disclosed herein. It is contemplated that outer layer 116 may be formed from a weaker material than inner layer 102. In some instances, the material of the outer layer 116 may only need to be strong enough to span holes 114. Outer layer 116 may be by casting, spraying, extrusion, molding, etc. In some embodiments, outer layer 116 may be formed directly on inner layer 102. In other embodiments, outer layer 116 may be formed as a separate structure and may be attached to the inner layer 102 by thermal or adhesive methods. It is contemplated that outer layer 116 may extend over the entire length of inner layer 102 or may extend along only a portion of the length of inner layer 102. For example, in some embodiments, outer layer 116 may be sized and shaped to cover the region of inner layer 102 including holes 114. As outer layer 116 is formed from a hydrophilic and/or conductive material, conductive regions, or virtual electrodes, may be defined in the regions of outer layer 116 adjacent to holes 114.

In use, balloon 100 may be used in a manner similar to balloon 22. For example, balloon 100 may be attached to catheter shaft such as catheter shaft 34 and used for a suitable intervention such as an ablation procedure. During ablation, a conductive fluid may be infused into balloon 100 and an electrode positioned within balloon 100 (e.g., electrode 24) may be activated. The energy may be transmitted through the medium of the conductive fluid and through conductive region adjacent to holes 114 to the blood vessel wall to modulate or ablate the tissue. Inner layer 102 may prevent the energy transmission through the balloon wall at locations other than conductive region.

Device 12 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming. In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of device 12 may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of device 12 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 12 to achieve the same result.

In some embodiments, a degree of Magnetic Resonance Imaging (MRI) compatibility is imparted into device 12. For example, device 12 or portions thereof, may be made of a material that does not substantially distort the image and create substantial artifacts (i.e., gaps in the image). Certain ferromagnetic materials, for example, may not be suitable because they may create artifacts in an MRI image. Device 12 or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers for device 12 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device for modulating nerves, the medical device comprising:
an elongate shaft having a distal region;
a balloon coupled to the distal region, the balloon having an inner non-conductive layer and an outer conductive layer;
an electrode disposed within the balloon; and
a virtual electrode defined along the balloon, the virtual electrode including a conductive region.

2. The medical device of claim 1, wherein the conductive region is defined along a section of the balloon that is free of the inner non-conductive layer.

3. The medical device of claim 1, wherein the conductive region is defined by one or more through holes formed in the inner non-conductive layer.

4. The medical device of claim 1, wherein the outer conductive layer covers the entire balloon.

5. The medical device of claim 1, wherein the outer conductive layer covers only a portion of the balloon.

6. The medical device of claim 1, wherein the electrode includes a coil electrode helically disposed about the shaft.

7. The medical device of claim 1, wherein the balloon includes a single virtual electrode.

8. The medical device of claim 1, further comprising one or more additional virtual electrodes.

9. The medical device of claim 1, further comprising a conductive fluid disposed within the balloon.

10. A medical device for modulating nerves, the medical device comprising:
an elongate shaft having a distal region and a fluid inlet and a fluid outlet proximate the distal region;
a balloon coupled to the distal region, the balloon having an inner non-conductive layer and an outer conductive layer;
an electrode disposed along the elongate shaft and positioned within the balloon; and
a virtual electrode defined along the balloon, the virtual electrode including a conductive region defined along a section of the balloon that is free of the inner non-conductive layer.

11. The medical device of claim 10, wherein the outer conductive layer covers the entire balloon.

12. The medical device of claim 10, wherein the outer conductive layer covers only a portion of the balloon.

13. The medical device of claim 10, wherein the conductive region includes one or more through holes in the inner non-conductive layer.

14. The medical device of claim 10, further comprising one or more additional virtual electrodes.

15. A method for manufacturing a medical device, the method comprising:
providing an expandable balloon formed of a non-conductive material;
forming one or more through holes in a portion the expandable balloon;
applying a conductive material over the portion of the expandable balloon including the one or more through holes;
wherein a virtual electrode is defined at a region adjacent to the one or more through holes.

16. The method of claim 15, wherein providing the expandable balloon includes extruding a tubular member and molding the expandable balloon.

17. The method of claim 15, wherein applying the conductive material includes bonding a thin tube to an outside surface of the expandable balloon.

18. The method of claim 15, wherein applying the conductive material includes spraying a conductive layer over the non-conductive material.

19. The method of claim 15, wherein applying the conductive material includes dipping the non-conductive material into the conductive material.

20. The method of claim 15, wherein the virtual electrode is defined along a first portion of the expandable balloon that is free of the non-conductive material.
